(19) United States
(12) Patent Application Publication
(10) Pub. No.: US 2023/0310660 A1
(43) Pub. Date: Oct. 5, 2023

(54) SHELF-STABLE INDOCYANINE GREEN SOLUTIONS AND METHODS OF MAKING THE SAME

(71) Applicant: CAO Group, Inc., West Jordan, UT (US)

(72) Inventor: Steven D. Jensen, South Jordan, UT (US)

(73) Assignee: CAO Group, Inc., West Jordan, UT (US)

(21) Appl. No.: 18/007,392

(22) PCT Filed: Jul. 28, 2021
(86) PCT No.: PCT/US21/43578

§ 371 Date: Jan. 30, 2023

Related U.S. Application Data
(60) Provisional application No. 63/059,440, filed on Jul. 31, 2020.

(51) Int. Cl. A61K 49/00

(52) U.S. Cl. CPC A61K 49/0034

(57) ABSTRACT

Shelf stable indocyanine green solutions are made by dissolving indocyanine green in an organic solvent with no water content. Such solutions may be self-stable for up to a year at room temperature when stored in air-tight containers. Solutions may then be used conventionally in imaging procedures in the medical and dental fields.

### CROSS-REFERENCES TO RELATED APPLICATIONS

The present invention claims priority as a non-provisional perfection of prior filed U.S. Application No. 63/059,440, filed on Jul. 31, 2020, and incorporates the same by reference herein in its entirety.

### FIELD OF THE INVENTION

The present invention relates to the field of dyes and more particularly relates to a stable form of the dye indocyanine green.

### BACKGROUND OF THE INVENTION

It is well known in the art that indocyanine green (ICG) solutions are unstable over a short shelf life of just a few days; for this reason, it is sold as a dry powder that is mixed and dissolved immediately prior to treatment. After mixing with water, ICG will degrade over days, or even just hours, to the point of being unusable.

Through experimentation, it has been determined that the biggest cause of instability within ICG solutions to be nothing other than water and the second most recognizable factor is temperature. Therefore, with the instability factors identified the present invention identifies by way of experimentation with other solutions within which ICG is stable. An embodiment of the present invention utilizes organic liquids to create shelf stable ICG solutions. The present invention is a method of creating a shelf-stable, liquid, indocyanine green solution.

### SUMMARY OF THE INVENTION

In view of the foregoing disadvantages inherent in the known types of ICG solutions, an improved shelf stable solution may utilize organic solvents to remove the instability found in prior art solutions. These solutions provide a self-stable ICG solution that will remain stable for up to one year.

The more important features of the invention have thus been outlined in order that the more detailed description that follows may be better understood and in order that the present contribution to the art may better be appreciated. Additional features of the invention will be described hereinafter and will form the subject matter of the claims that follow.

Many objects of this invention will appear from the following description and appended claims, reference being made to the accompanying drawings forming a part of this specification wherein like reference characters designate corresponding parts in the several views.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also, it is to be understood that the phraseology and terminology employed herein are for description and should not be regarded as limiting.

As such, those skilled in the art will appreciate that the conception, upon which this disclosure is based, may readily be utilized as a basis for the designing of other structures, methods, and systems for carrying out the several purposes of the present invention. It is important, therefore, that the claims be regarded as including such equivalent constructions insofar as they do not depart from the spirit and scope of the present invention.

### DETAILED DESCRIPTION OF THE PREFERRED EMBODIMENT

With reference now to the drawings, a preferred embodiment of the shelf stable indocyanine green solutions is herein described. It should be noted that the articles “a”, “an”, and “the”, as used in this specification, include plural referents unless the content clearly dictates otherwise.

A preferred embodiment of the present invention utilizes organic liquids that contain no additional water. An embodiment of the present invention utilizes absolute mono-alcohols such: ethyl, methyl, isopropyl, n-propyl, butyl alcohols and any other alcohols that are liquids. An embodiment of the present invention utilizes poly-ols such: glycerin, propylene glycol, polyethylene glycols and any other poly-ols that are liquids. An embodiment of the present invention utilizes mixtures of multiple organic solvents to customize particular characteristics for the delivery of soluble ICG such as: viscosity, capillary action, water chasing efficiency and other characteristics.

In the absence of water, it has been found that indocyanine green dissolved in organic solvent is shelf stable for up to 1 year at room temperature. More importantly, the type of organic solvent selected is not the important factor for stability—only the absence of water is required.

When creating the solution, the manufacturing method is to add the ICG to the solvent(s) and stir until it dissolves. Thereafter the solution is loading in an airtight sealed container, thereby preventing the introduction of ambient air, which may contain water vapor, into the container.

### INDUSTRIAL APPLICABILITY

The present invention has industrial applicability in that it may be made in industry and has particular applicability for imaging in the medical and dental fields.

Although the present invention has been described with reference to preferred embodiments, numerous modifications and variations can be made and still the result will come within the scope of the invention. No limitation with respect to the specific embodiments disclosed herein is intended or should be inferred.

1. (canceled)

2. (canceled)

3. (canceled)

4. A method of making a solution of indocyanine green comprising:
a first step of adding indocyanine green powder to an organic solvent having no water content;
a second step of mixing the indocyanine green and the solvent until the indocyanine green dissolves; and
a third step of storing the solution in an airtight, sealed container until use of the solution.

5. The method of claim 4, the solution being stored for a time period of up to one year.
